(19) United States
(12) Patent Application Publication
(10) Pub. No.: US 2007/0073315 A1
(43) Pub. Date: Mar. 29, 2007

(54) Systems And Methods For Closing Internal Tissue Defects

(76) Inventors: Richard Ginn, Gilroy, CA (US); Joseph Karratt, Millbrae, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 11/558,298

(22) Filed: Nov. 9, 2006

Related U.S. Application Data
(62) Division of application No. 10/847,747, filed on May 17, 2004.

(51) Int. Cl. A61B 17/08

(52) U.S. Cl. 606/151

(57) ABSTRACT

A delivery system having a steerable delivery device and an elastic clip for treating an internal tissue defect, such as septal defects and the like, is provided. The clip can be deformable between a relaxed state and a stressed state and biased towards the relaxed state. The delivery device can include a flexible tubular needle with a lumen for housing the clip while in the stressed state. When used to treat a septal defect, the needle is advancable through overlapping tissue flaps and a pusher member is advancable within the needle lumen to push the clip distally such that one end of the clip is deployed and engages a tissue flap. The needle can then be retracted allowing the clip to deploy over both flaps of tissue, where the biasing force returns the clip to the relaxed state drawing the tissue flaps together and closing the septal defect.

### CROSS-REFERENCE TO RELATED APPLICATIONS

This application is a divisional of U.S. patent application Ser. No. 10/847,747, filed on May 17, 2004, which is fully incorporated by reference herein.

### FIELD OF THE INVENTION

The present invention relates generally to systems and methods for closing internal tissue defects, and more particularly to systems and methods for closing a patent foramen ovale or other defect with a deformable elastic clip.

### BACKGROUND OF THE INVENTION

By nature of their location, the treatment of internal tissue defects is inherently difficult. Access to a defect through invasive surgery introduces a high level of risk that can result in serious complications for the patient. Access to the defect remotely with a catheter or equivalent device is less risky, but treatment of the defect itself is made more difficult given the limited physical abilities of the catheter. The difficulty in accessing and treating tissue defects is compounded when the defect is found in or near a vital organ. For instance, a patent foramen ovale (“PFO”) or patent ductus arteriosus (“PDA”), is a serious septal defect that can occur between the left and right atria of the heart.

During development of a fetus in utero, blood is oxygenated by the mother's placenta, not the fetus' developing lungs. Most of the fetus' circulation is shunted away from the lungs through specialized vessels or foramens that are open during fetal life, but close shortly after birth. Occasionally, however, these foramen fail to close and create hemodynamic problems, which can ultimately prove fatal. During fetal life, an opening called the foramen ovale allows blood to pass directly from the right atrium to the left atrium (bypassing the lungs). Thus, oxygenated blood from the placenta may travel through the vena cava into the right atrium, through the foramen ovale into the left atrium, and from there into the left ventricle for delivery to the fetus' body. After birth, with pulmonary circulation established, the increased left atrial blood flow and pressure causes the functional closure of the foramen ovale and, as the heart continues to develop, this closure allows the foramen ovale to grow completely sealed.

In some cases, however, the foramen ovale fails to close entirely. This condition, known as a patent foramen ovale, can pose serious health risks for the individual, particularly if the individual has other heart abnormalities. For example, recent studies suggest an association between the presence of a patent foramen ovale and the risk of paradoxical embolism or stroke. See P. Lechat J et al., “Prevalence of Patent Foramen ovale in Patients with Stroke,” N. Engl. J. Med. 1988;318: 1148-1152.

Still other septal defects can occur between the various chambers of the heart, such as atrial-septal defects (ASD's), ventricular-septal defects (VSD's), and the like. To treat such defects, open heart surgery can be performed to ligate and close the defect. Alternatively, catheter-based procedures have been developed that require introducing umbrella or disc-like devices into the heart. These devices include opposing expandable structures connected by a hub or waist. Generally, in an attempt to close the defect, the device is inserted through the defect and the expandable structures are deployed on either side of the septum to secure the tissue surrounding the defect between the umbrella or disc-like structure. Such devices, however, involve frame structures that often support membranes, either of which may fail during the life of the patient. Thus, the treatment of septal defects with these devices introduces the risk that the defect may reopen or that portions of the device could be released within the patient's heart.

Accordingly, improved systems and methods for closing internal tissue defects such as patent foramen ovale, patent ductus arteriosus and other septal and tissue defects are needed.

### SUMMARY

Improved systems and methods for closing internal tissue defects, such as septal defects and the like, are provided herein. Preferably, a delivery device is used to place an elastic clip over the defect, such that the elastic clip can at least partially close and preferably seal the defect with minimum risk to the patient. In one exemplary embodiment, the elastic clip has a first end, a second end and a body therebetween, where the body has a longitudinal axis extending along its length. The clip is preferably biased towards a relaxed state where the ends are adjacent to each other, for instance, in a ring-like shape, wherein upon application of a mechanical stress the clip is deformable from the relaxed state to a stressed state. In the stressed state the body can be straightened such that each end extends in a direction at least partially away from the other placing the body in torsion about the longitudinal axis. In another embodiment, the clip can have multiple coiled segments located adjacent to each other, where preferably at least one of which forms a 360 degree loop around at least one axis of the clip while in the relaxed state.

Also provided is a steerable delivery device for delivering the elastic clip to the tissue defect. In one exemplary embodiment, the steerable delivery device includes a flexible elongate tubular body having a distal end with an opening therein, a proximal end and an inner lumen, a flexible elongate tubular needle having a sharp, open distal end, a proximal end and an inner lumen, with the needle being slidable within the inner lumen of the body. The device also includes a flexible elongate pusher member having a distal end and a proximal end, with the pusher member being slidable within the inner lumen of the needle, wherein the opening in the distal end of the body is adapted or sized to allow the needle to pass therethrough. To provide steerability, the device can include a wire coupled with the distal end of the body and extending proximally along the body, in addition to a bias member housed within the body. The bias member can be configured to apply a bias to the body along a longitudinal axis of the body. Preferably, the wire is configured to bend the device upon application of a force to the wire in a proximal direction, allowing the distal end of the device to be steered into proximity with the tissue defect, as well as allowing the device to be steered through the patient's vasculature or other body cavities, if desired. An actuator can be provided on the proximal end of the device for controlling the movement of the needle, pusher member and/or wire.

Also provided is a method for closing a tissue defect, such as a septal defect, with a delivery device and elastic clip. In one preferred embodiment of the method, the delivery device is advanced into proximity with the septal defect, the device having a flexible elongate tubular body with an inner lumen and a distal end with an opening therein. A flexible elongate tubular needle having an inner lumen and a sharp, open distal end, is then slidably advanced from within the inner lumen of the body such that the needle pierces and penetrates a first and a second tissue flap of the septal defect. Preferably, a flexible elongate pusher member is housed within the inner lumen of the needle, the pusher member having a distal end in contact with an elastic clip also housed within the inner lumen of the needle. The pusher member is slidably advanced to deploy a first end of the elastic clip from the open distal end of the needle. The needle is then retracted from the tissue flaps such that the clip is deployed over the tissue flaps where it can at least partially close an opening therebetween.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims. It is also intended that the invention is not limited to the require the details of the example embodiments.

### BRIEF DESCRIPTION OF THE FIGURES

The details of the invention, both as to its structure and operation, may be gleaned in part by study of the accompanying figures, in which like reference numerals refer to like parts. The components in the figures are not necessarily to scale, emphasis instead being placed upon illustrating the principles of the invention. Moreover, all illustrations are intended to convey concepts, where relative sizes, shapes and other detailed attributes may be illustrated schematically rather than literally or precisely.

FIG. 1A is an axial cross-sectional view of one exemplary embodiment of a delivery system.

FIG. 1B is an radial cross-sectional view of the delivery system taken along B-B of FIG. 1A.

FIG. 1C is an axial cross-sectional view of another exemplary embodiment of the delivery system while in a deflected state.

FIGS. 2A-C are exterior views of exemplary embodiments of elongate flexible tubular needles for use in the delivery system.

FIG. 3 is an axial cross-sectional view of another exemplary embodiment of the delivery system.

FIG. 4 is an axial cross-sectional view of another exemplary embodiment of the delivery system.

FIGS. 5A-E are axial cross-sectional views depicting the delivery of an exemplary embodiment of an elastic clip to a septal defect with an exemplary embodiment of the delivery system.

FIG. 5F is a perspective view of the septal defect shown in FIGS. 5A-E closed by the elastic clip after deployment from the delivery system.

FIG. 6A is a top-down view of an exemplary embodiment of the elastic clip while in the relaxed state.

FIGS. 6B-D are perspective views of an exemplary embodiment of the elastic clip, where: FIG. 6B depicts the clip while in the relaxed state; FIG. 6C depicts the clip while in a partially stressed state; and FIG. 6D depicts the clip while in the stressed state.

FIG. 7 is a top-down view of another exemplary embodiment of the elastic clip.

FIG. 8A is a perspective view of an exemplary embodiment of a coiled elastic clip while in the relaxed state.

FIG. 8B is a perspective view of the coiled elastic clip of FIG. 8A while in the stressed state.

FIG. 9 is a perspective view of another exemplary embodiment of the coiled elastic clip while in the relaxed state.

FIG. 10A is a perspective view of another exemplary embodiment of the coiled elastic clip while in the relaxed state.

FIG. 10B is a perspective view of the coiled elastic clip of FIG. 10A in a partially stressed state.

FIG. 11A is a perspective view of another exemplary embodiment of the coiled elastic clip while in the relaxed state.

FIG. 11B is a perspective view of the coiled elastic clip of FIG. 11A in the stressed state.

### DETAILED DESCRIPTION

The systems and methods described herein provide a deformable elastic clip and a steerable delivery device for use in the treatment of internal tissue defects. Preferably, these systems and methods are used to treat a septal defect where an undesired opening allows blood to shunt within the heart. Examples of such defects include PFO's, PFA's, ASD's, VSD's and the like. Frequently, the opening in the septum is surrounded by overlapping flaps of tissue that have failed to close properly. The steerable delivery device can be used to steer the distal end of the delivery device into proximity with the tissue defect and position the clip in close proximity to these tissue flaps. Once in position, the steerable delivery device can deposit the elastic clip over the flaps such that the clip can draw the flaps together and at least partially close or seal the opening. The clip can then remain engaged to the tissue for an indefinite period of time, holding the defect closed and giving the tissue the opportunity to form together and properly seal itself.

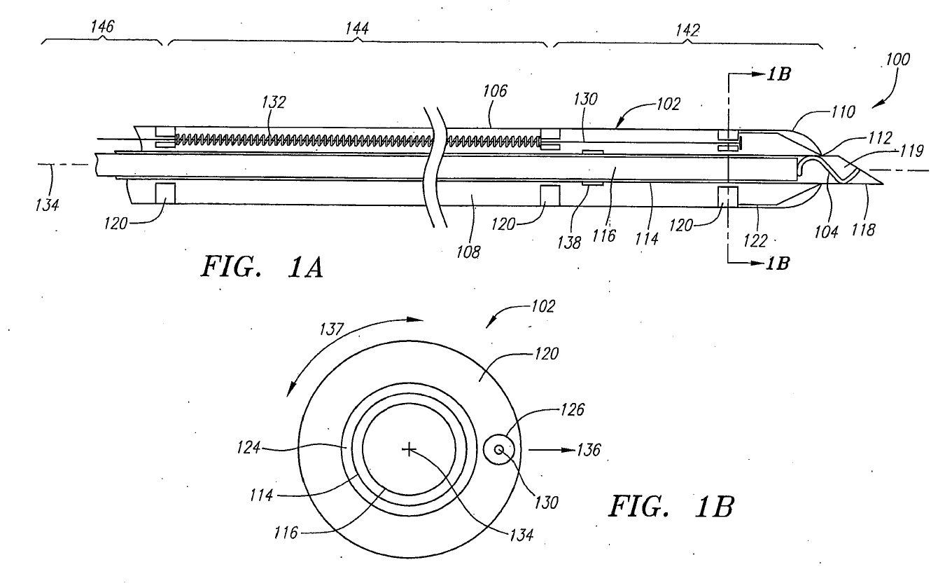

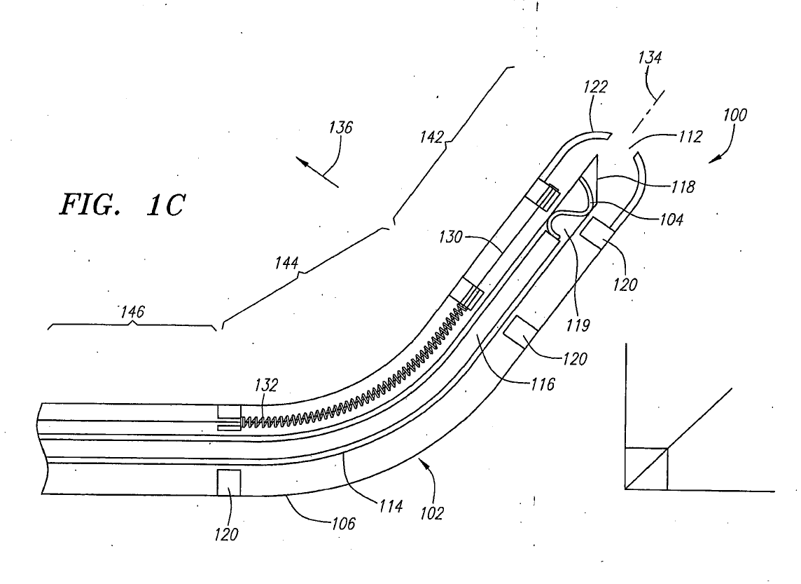

FIG. 1A depicts an axial cross-sectional view of a preferred exemplary embodiment of delivery system 100, which is used to treat an internal tissue defect. Delivery system 100 includes delivery device 102 and elastic clip 104. Delivery device 102 is preferably used to navigate within the patient's body and deliver clip 104 to the tissue defect. Delivery device 102 can be configured as, or integrated with, any medical device suitable for internal medical procedures, such as a catheter, endoprobe and the like. In this embodiment, delivery device 102 is depicted as an intravascular catheter. Clip 104 is deformable from a relaxed state to a stressed state upon application of a mechanical stress. When this stress is removed, clip 104 preferably returns to the relaxed state. Here, clip 104 is shown housed within delivery catheter 102 while in the stressed state. Clip 104 can be delivered from catheter 102 over the tissue defect such that clip 104 at least partially closes the defect, and preferably seals the defect, as it returns to the relaxed state.

In this embodiment, catheter 102 includes a flexible elongate tubular body 106 having inner lumen 108 therein. Body 106 is preferably formed from a high-durometer, e.g., 55D, material, but is not limited to such and can vary with the needs of the application. Catheter 102 also includes flexible elongate tubular needle 114, which is configured to slide within inner lumen 108. Needle 114 has sharpened, open distal end 118, as well as inner lumen 119, which can be sized to house clip 104. The distal end 110 of body 106 preferably has a tapered or rounded tip for facilitating atraumatic advancement of catheter 102 through the patient's body. Here, distal end 110 has a rounded, rigid distal tip 122 with opening 112 therein. Opening 112 is preferably sized to allow needle 114 to slide therethrough, while tip 122 is rigid in order to prevent needle 114 from piercing body 106.

Additionally, catheter 102 includes flexible pusher member 116, which also has an elongate shape and is configured to slide within inner lumen 119 of needle 114. Pusher member 116 can be optionally configured as a second catheter, with imaging or diagnostic capabilities and the like. Needle 114 and pusher member 116 are preferably formed from nitinol, but are not limited to such and can be formed from any material in accordance with the needs of the application.

Here, delivery system 100 includes three sections: distal section 142; intermediate section 144; and proximal section 146. Intermediate section 144 is located proximal to distal section 142, while proximal section 146 is located proximal to intermediate section 144. These sections are included in FIG. 1A only to aid in further illustrating the operation of delivery system 100 and are not intended to limit the systems and methods described herein.

In order to provide steerability to delivery system 100, catheter 102 includes deflection wire 130 and bias member 132 extending longitudinally within catheter body 106. Deflection wire 130 is coupled with guide member 120 at the distal end 110 of catheter 102 and extends proximally along the length of catheter body 106. Wire 130 can be coupled with guide member 120 in any manner and is shown here having a widened distal tip, which catches guide member 120 and prevents wire 130 from passing proximally through lumen 126 (shown in more detail in FIG. 1B).

Bias member 132 is housed within catheter 102 and located along intermediate section 144, preferably between guide members 120. In this embodiment, each end of bias member 132 abuts a guide member 120 and applies a bias force between them, although any abutment within catheter body 106 can be used. In one embodiment, a notch or receiving hole is placed in guide members 120 to abut bias member 132 and maintain bias member 132 in place. In a preferred embodiment, bias member 132 is a stacked teflon-coated coil spring having a round cross-section and placed over deflection wire 130. However, any type, shape or configuration of bias member 132 can be used according to the needs of the application.

As shown in FIG. 1A, catheter 102 can include one or more guide members 120. FIG. 1B depicts a radial cross section of catheter 102 taken along B-B of FIG. 1A. In this exemplary embodiment, each guide member 120 includes needle lumen 124 and deflection wire lumen 126, used for guiding the motion of needle 114 and deflection wire 130, respectively. In another embodiment, each guide member 120 can include one single lumen sized to fit both the needle 114 and deflection wire 130. Although three guide members 120 are shown here, any number of guide members 120 can be placed at any desired positions along the length of catheter body 106 as needed by the application.

Deflection wire 130 is preferably located along a longitudinal axis of catheter 102 that is offset from central axis 134. When a force is applied to wire 130 in a proximal direction, i.e., by “pulling” wire 130, the distal end 110 of catheter 102 deflects in radial direction 136, which is depicted in FIG. 1B. The deflection of distal end 110 in direction 136 preferably causes catheter body 106 to bend, mainly within intermediate section 144 such that this portion of catheter 102 acts as an “elbow.” FIG. 1C depicts an axial cross-sectional view of catheter 102 with distal end 110 deflected at an angle of 45 degrees. When wire 130 is released, bias member 132 causes distal end 110 to return from the deflected state to the original, straightened state. The catheter body 106 preferably includes a relatively stiff material to reinforce catheter 102 and prevent buckling. In one embodiment, a metallic braided shaft is integrated with catheter body 106 along section 144 to provide reinforcement.

Catheter 102 can be steered in any direction within a patient's body in order to properly position distal end 110 in proximity with the tissue defect, or in order to navigate through the patient's vasculature. Because pulling deflection wire 130 will deflect distal end 110 in direction 136, catheter 102 may first require axial rotation in direction 137 to properly orient catheter 102. For example, while steering catheter 102 into proximity with the tissue defect, it may be desirable to deflected distal end 110 first to the left and then to the right in order to properly position catheter 102. In this case, after deflecting distal end 110 of catheter 102 to the left, the user would rotate catheter 102 by 180 degrees to properly orient catheter 102 before pulling wire 130 to deflect distal end 110 to the right. The distance the user pulls deflection wire 130 back determines the amount of deflection in the distal end 110. For instance, a deflection of 90 degrees would require more pull back than a deflection of 45 degrees. In this manner, distal end 110 can be properly positioned in proximity with the defect.

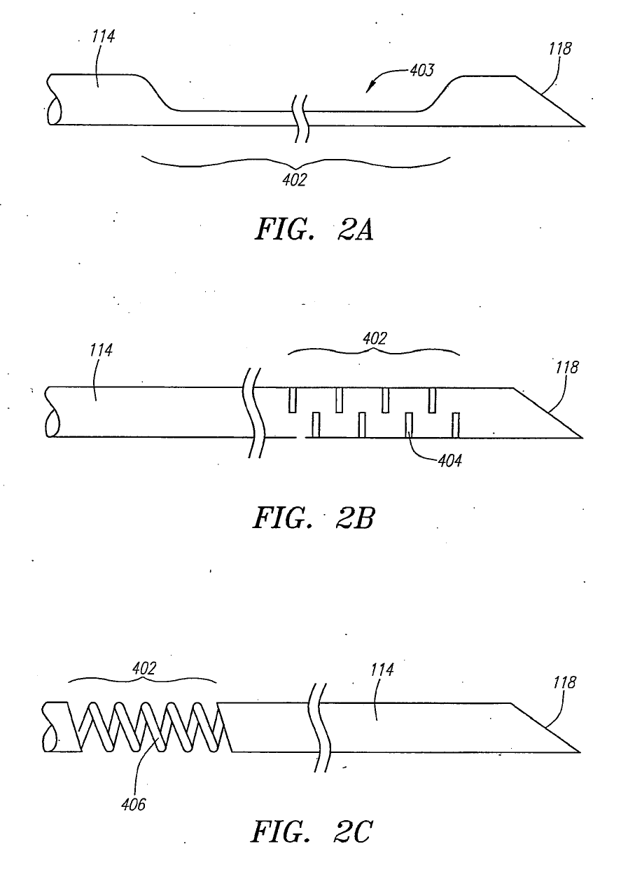

FIGS. 2A-C depict exemplary embodiments of needle 114 for use with steerable embodiments of delivery catheter 102 such as that depicted in FIG. 1A. These embodiments of needle 114 have a region configured to provide increased flexibility during deflection. FIG. 2A depicts one embodiment of needle 114 having flexible region 402, which, in this instance, is an open portion 403 in the tubular body of needle 114 that reduces the stiffness of needle 114. Flexible region 402 is preferably positioned on needle 114 such that region 402 is within intermediate region 144, i.e., the elbow portion of catheter 102 during deflection. In applications where needle 114 is advanced or retracted while catheter 102 is in a deflected state, flexible region 402 is preferably present over an axial length of needle 114 longer than the axial length of intermediate region 144 so that flexible region 402 is not advanced or retracted from the bent portion of catheter 102.

Flexible region 402 can be formed in needle 114 using various differing methods including electrical discharge machining (EDM), laser cutting, photolithography any type of patterning and the like. In addition, any desired pattern can be formed in flexible region 402. FIG. 2B and FIG. 2C depict additional exemplary embodiments of needle 114 having various flexible regions 402. In FIG. 2B, flexible region 402 includes multiple slot-shaped apertures 404 circumscribing needle 114. In FIG. 2C, flexible region 402 includes a coil-shape, or helically-wound segment, in needle 114 to provide added flexibility. This coiled region 406 can optionally be used in place of bias member 132, for instance, in order to return catheter 102 from a deflected state to a straightened state.

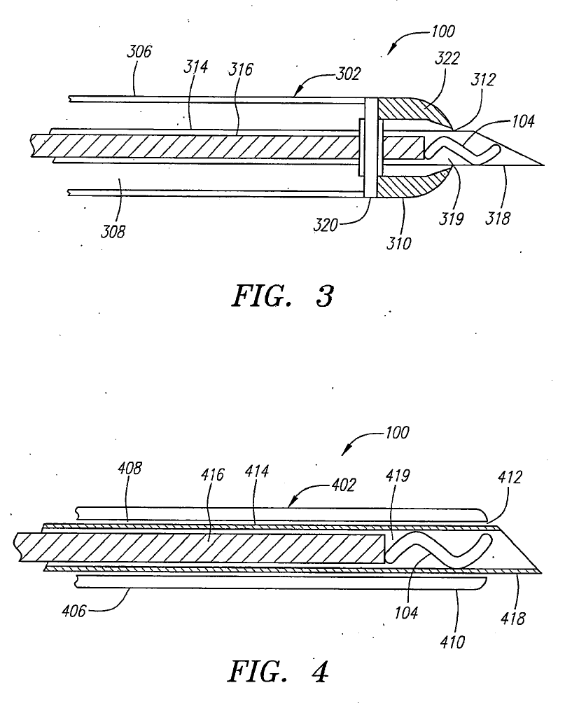

FIG. 3 and FIG. 4 depict additional exemplary embodiments of delivery system 100. These embodiments lack the deflection wire 130 and bias member 132 shown in the embodiment of FIGS. 1A-B. In FIG. 3, delivery system 100 includes catheter 302, which includes flexible needle 314 and flexible pusher 316 for delivering elastic clip 104 to a tissue defect. Catheter 302 includes rigid distal tip 322 to prevent needle 314 from piercing body 306 and can also include one or more guide members 320 for guiding the sliding motion of needle 314 within inner lumen 308. In FIG. 4, delivery system 100 includes catheter 402 having an inner lumen 408, which provides relatively less space between needle 414 and body 406 than the previous embodiments shown in FIGS. 1A-B and FIG. 3. In this embodiment, catheter 402 lacks rigid distal tip 322 and, thus, distal end 410 of catheter body 406 is more susceptible to being inadvertently pierced by needle 414. Catheter 402 can be sized relatively small and is therefore preferably used to navigate through more narrow vasculature.

Although not shown, delivery system 100 can be used with pre-shaped members, such as a pre-shaped body, needle, pusher member, deflection wire and the like. For instance, in one exemplary embodiment, needle 114 has a 90 degree pre-shaped curve near the distal end. Needle 114 can then be used in much the same way as a stylet, where insertion of needle 114 into inner lumen 108 of body 106 deflects catheter 102. Also, additional pre-shaped members can be used to counter other pre-shaped members. For instance, a pre-shaped pusher member 116 can have a 90 degree bend near the distal end, and can be inserted into lumen 119 of needle 114 such that the bend is oriented in the opposite direction. Thus, when pusher member 116 is fully advanced within needle 114 the opposing bends counteract each other and straighten catheter 102.

As discussed above, the systems and methods described herein can be used to treat numerous types of tissue defects including septal defects and the like. For ease of illustration, the following embodiments are described in the context of treating one particular type of defect, namely a PFO. However, it should be noted that although the following discussion takes place in this exemplary context, the systems and methods described herein are not limited solely to the treatment of a PFO and can in fact be extended to a wide variety of tissue defects.

To treat a PFO, catheter 102, with clip 104 housed therein, can be introduced into the patient's vasculature, e.g., from a percutaneous entry site in a peripheral vessel, such as the femoral vein, jugular vein and the like. Distal end 110 of catheter 102, including clip 104, can be advanced endoluminally within the patient's vasculature, e.g., through the vena cava (inferior or superior) and into the heart until distal end 110 is disposed within the a heart chamber, such as the right atrium. Alternatively, clip 104 can be introduced using an arterial approach as is commonly known in the art.

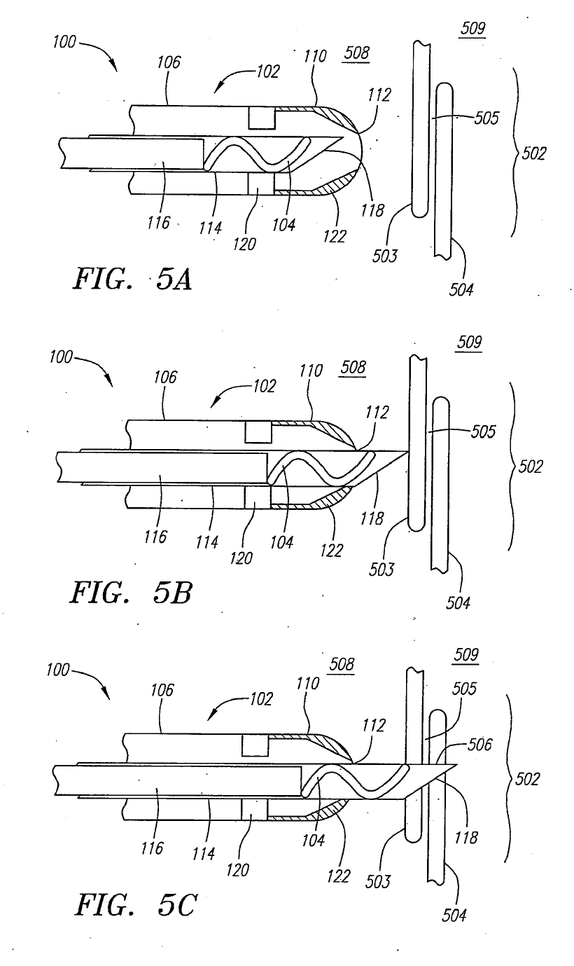

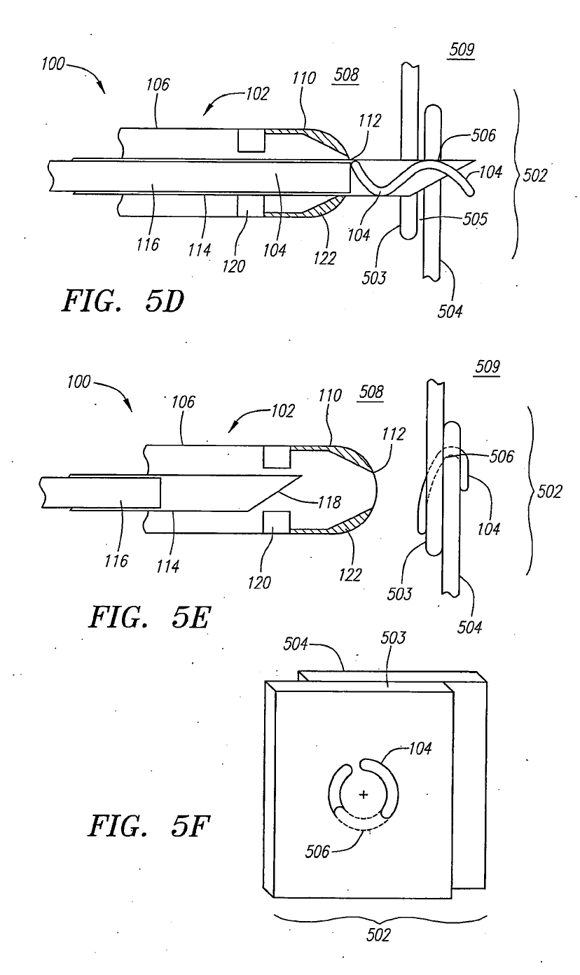

Catheter 102 is then navigated into proximity with PFO region 502, as depicted in FIG. 5A. Accordingly, catheter 102 can include an imaging device (not shown), such as an ultrasound transducer or optical imager, to aid navigation through the patient's vasculature and body cavities. The imaging device can be placed at or near distal end 110 of catheter 102, e.g., attached to or adjacent distal tip 122 or advanceable from lumen 108. In a further alternative, external imaging may be used, either alone or in conjunction with direct visualization. For example, clip 104, catheter body 106, needle 114 and/or pusher member 116 can include radio opaque markers at predetermined locations that can be observed using fluoroscopy and the like. Referring back to FIG. 1A, catheter 102 is shown having radio opaque marker 138 on needle 114, which can be a platinum-iridium (PT-IR) ring and the like, to enable the user to visually locate catheter 102 within the patient's body.

In one preferred embodiment, a guiding catheter (not shown) is first navigated into proximity with PFO region 502 and catheter 102 is then advanced within the guiding catheter. The guiding catheter can include an imaging device or it can be guided into place using other external imaging methods. Once catheter 102 is in position, needle 114 can be advanced distally from opening 112 into contact with PFO region 502, as depicted in FIG. 5B. PFO region 502 is defined by two overlapping tissue flaps 503 and 504 with an undesired opening 505 therebetween. Needle 114 pierces the tissue flaps 503 and 504 in the PFO, creating aperture 506 as depicted in FIG. 5C.

Once open distal end 118 of needle 114 has penetrated both tissue flaps 503 and 504, pusher 116 can be advanced distally within inner lumen 119 of needle 114 until one end of clip 104 protrudes from open distal end 118 and engages side 509 of tissue flap 504 as depicted in FIG. 5D. Needle 114 can then be retracted proximally so that clip 104 is deployed within aperture 506 and over tissue flaps 503 and 504 as depicted in FIG. 5E. As clip 104 is deployed from within needle 114, the mechanical stress keeping clip 104 in the stressed state is removed. Thus, as clip 104 is deployed it begins to return to the relaxed state. When properly deployed over tissue flaps 503 and 504, this return to the relaxed state draws flaps 503 and 504 together, at least partially closing and preferably sealing opening 505, as depicted in the perspective view of FIG. 5F.

In order to deploy clip 104, the user preferably holds pusher 116 in a static position relative to PFO region 502 while retracting needle 114 proximally over pusher 116. This maintains clip 104 in the proper position relative to tissue flaps 503 and 504, so that upon removal of needle 114, clip 104 properly closes opening 505. Alternatively, clip 104 can be delivered by allowing one end of clip 104 to engage tissue flap 504 on side 509 after being advanced from distal end 118, i.e., “catching” tissue flap 504 so that clip 104 remains in place as needle 118 is retracted from aperture 506. In this manner, clip 104 is deployed over PFO region 502 regardless of whether pusher 116 is held in a static position.

In order to facilitate the deployment of clip 104 by the user, an actuator, e.g., a handle device (not shown), can be provided on the proximal end of catheter 102. The actuator preferably permits controlled advancement of both needle 114 and pusher member 116 as well as relative movement between them. For example, the actuator can allow the distal end of the pusher member 116 to be disposed at a location within or external to sharpened distal end 118 of needle 114. The actuator can also provide controls to the amount of movement of needle 114, for instance, to prevent needle 114 from advancing too far past the tissue flaps prior to deploying clip 104.

Although not shown, the inner surface of needle 114 can optionally include one or more axially disposed grooves to guide the movement of clip 104. The groove(s) can maintain clip 104 in a relatively fixed radial orientation during advancement of catheter 102 through the patient's body and also during delivery, so that clip 104 does not rotate into a different orientation. Optionally, the distal end of pusher member 116 can have a notch or indentation (not shown), which engages with one end of clip 104 for assisting in the orientation of clip 104. The notch or indentation can prevent the rotation of clip 104, or alternatively, aid in rotating clip 104 through rotation of pusher member 116. The notch or indentation can be present without or in addition to any axial groove(s).

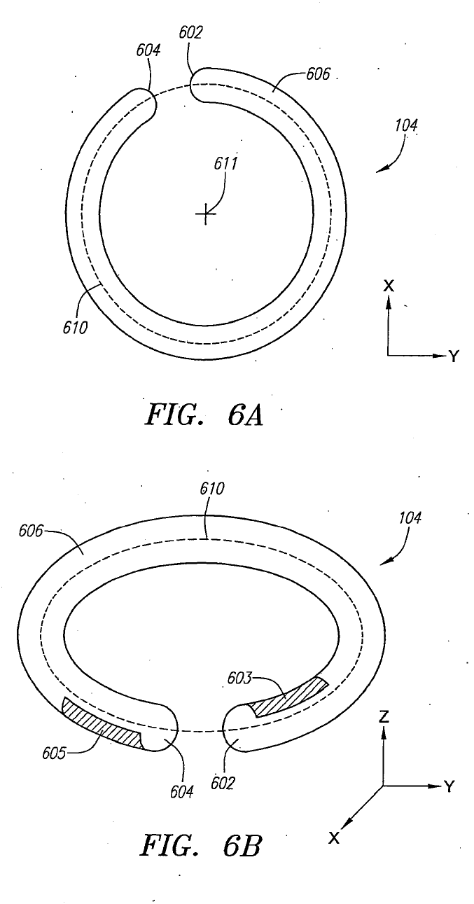

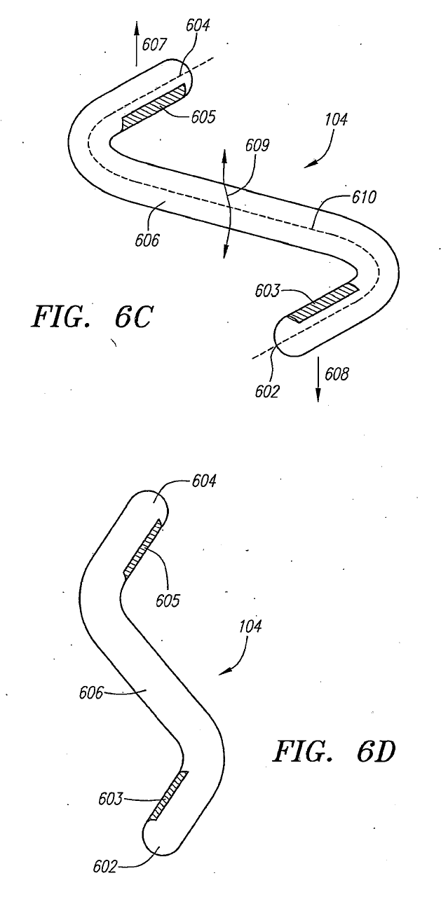

FIG. 6A depicts a top down view of one exemplary embodiment of clip 104, for use with the systems and methods described herein. Elastic clip 104 includes body 606 with ends 602 and 604 extending along longitudinal axis 610. Here, clip 104 is curved in a generally circular, ring-like shape around central axis 611. Ends 602 and 604 are preferably atraumatic or substantially blunt, i.e., shaped to minimize trauma to the internal tissue of the patient. Ends 602 and 604 can be tapered or rounded, as depicted here. Clip 104 can be formed from an elastic material, such as stainless steel, and preferably a superelastic material having shape memory and superelastic characteristics, such as nitinol, various nitinol alloy combinations and the like. The shape memory material can be pre-processed in the relaxed state in order to provide the material with memory of the relaxed state shape. The pre-processing of materials such as nitinol to instill shape memory and superelastic characteristics is well known to one of skill in the art. Of course, bio-degradable materials can also be employed in the formation of clip 104.

FIGS. 6B-D depict perspective views of one exemplary embodiment of deformable elastic clip 104. Shaded regions 603 and 605 denote the surface portion of clip 104, which engages each tissue flap. In order to more adequately engage the tissue flaps, shaded regions 603 and 605 can have a roughened surface texture that increases the frictional resistance when in contact with the tissue flaps. In should be noted that the position of shaded regions 603 and 605 can vary depending on the layout and shape of clip 104, as well as the type of tissue defect being treated.

In a preferred embodiment, clip 104 is biased towards a relaxed state as shown in FIG. 6B, where ends 602 and 604 are adjacent to one another and at least partially oppose each other. Clip 104 is deformable from the relaxed state to a stressed state upon application of a mechanical stress. FIG. 6C shows clip 104 deformed partially towards the stressed state where a stress applied between ends 602 and 604 in directions 607 and 608 moves each end 602 and 604 laterally away from the other. This deformation preferably twists body 606 and places body 606 in torsion around longitudinal axis 610, as indicated by arrow 609. This torsional force biases clip 104 towards the relaxed state such that clip 104 returns to the relaxed state upon removal of the mechanical stress, i.e., upon delivery from needle 114. As will be discussed below, clip 104 can be configured such that the biasing force is only exhibited when the clip is above a transitional temperature.

While in the relaxed state, clip 104 can rest substantially within the X-Y plane. However, the layout of clip 104 while in the relaxed state can vary with the needs of the application. For instance, in some applications it can be desirable for ends 602 and 604 to be partially deflected away from each other in the Z direction, in a direction opposite directions 607 and 608. This increases the amount of deformation needed to place clip 104 in the stressed state and, depending on the materials employed in forming clip 104, can result in a stronger return force generated by clip 104 when returning from the stressed state to the relaxed state.

FIG. 6D depicts clip 104 in the fully stressed state where each end 602 and 604 is deformed from the substantially planar relaxed state of FIG. 6A. Here, ends 602 and 604 extend along longitudinal axis 610 at least partially away from each other and body 606 is straightened in the Z-direction. This straightened state allows clip 104 to be readily housed within inner lumen 119 and delivered through the PFO such that ends 602 and 604 can engage opposite sides of the tissue flaps.

Depending on the type and nature of the tissue defect, clip 104 can have many variations in design. Notably, clip 104 can have any shape as desired for use in the application. For instance, clip 104 can have a curved shape such as circular, ring-like, arcuate, elliptical, oval or eccentric, or clip 104 can have a multi-sided shape such as square, rectangular, hexagonal or pentagonal, or clip 104 can have any combination of shapes. Clip 104 can also be shaped symmetrically or asymmetrically. The length, width and cross-sectional shape of clip 104 can be chosen depending on the thickness of the tissue flaps. Also, the relative position of ends 602 and 604 in the relaxed state and stressed state can vary according to the amount of closing strength needed to close the tissue flaps. As mentioned above, the material characteristics of clip 104 can also be varied. In one embodiment, clip 104 can be formed from a bio-degradable material degrading over a length of time sufficient to allow the tissue flaps to seal themselves.

Clip 104 can also be composed of nitinol and configured to have an Austenite finish (Af) temperature close to that of the human body temperature. Thus, while in the Martensitic phase outside of the body, clip 104 can be deformed to the stressed state and readily loaded into needle 114. After clip 104 is placed within the body, it is heated past the Af temperature and changes to the Austensitic phase where clip 104 becomes biased towards the relaxed state. The ability of clip 104 to be configured such that it does not experience the biasing force when below the Af temperature, makes it easier, from a practical standpoint, for clip 104 to be placed in a wide variety of different stressed states. For instance, clip 104 can be straightened entirely with no curves or bends. This would then allow clip 104 to be used in a relatively smaller catheter 102 in relatively smaller anatomies.

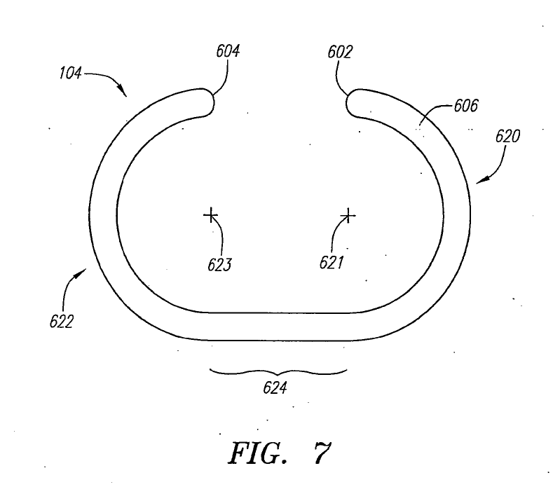

In FIGS. 6A-D, clip 104 is shown curved around one central axis 611 with ends 602 and 604 adjacent to each other. Clip 104 can be shaped or curved around one or more different axes. FIG. 7 depicts a top view of one exemplary embodiment of clip 104 curved in an eccentric ring-like shape while in the relaxed state. Here, a first portion 620 of clip 104 is curved around first axis 621, and a second portion 622 of clip 104 is curved about second axis 623, and a substantially straight, extended midsection 624 is located between portions 620 and 622. This embodiment is one example of a configuration that can be used when the tissue flaps are relatively thick with extended midsection 624 allowing greater engagement of the tissue flaps by portions 620 and 622.

In the embodiments depicted in FIGS. 6A-D, body 606 is generally circular in a radial cross-section, i.e., a cross-section taken along a plane having longitudinal axis 610 as a normal. However, clip 104 is not limited to a circular cross-section and can have any desired cross-sectional shape. In one exemplary embodiment, clip 104 can have an elliptical cross-section, while in another exemplary embodiment, clip 104 can have an rectangular cross-section with at least one side longer then the others, which can be roughened to more adequately engage the tissue flaps.

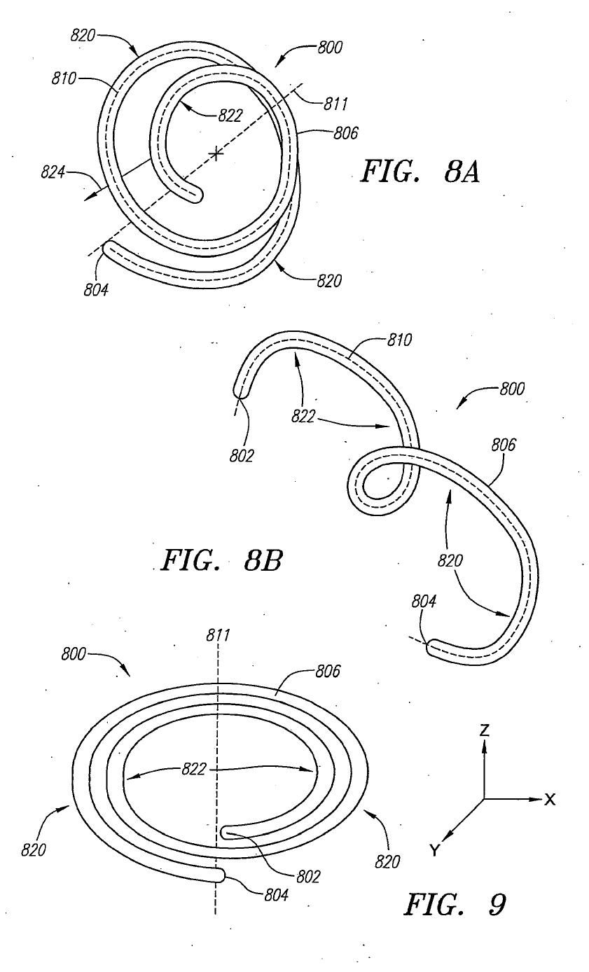

FIG. 8A depicts a perspective view of another exemplary embodiment of clip 800, for use with the systems and methods described herein as an alternative to clip 104. Elastic clip 800 includes coiled body 806 with adjacent ends 802 and 804 opposing each other and extending along longitudinal axis 810. While in the relaxed state, this embodiment of clip 800 is coiled in a helical shape around central axis 811. Clip 800 has first coiled segment 820 and second coiled segment 822, each preferably looping 360 degrees about central axis 811. Second coiled segment 822 preferably has a smaller perimeter than first coiled segment 820. When clip 800 is deformed from the relaxed state to the stressed state, second coiled segment 822 is preferably passed within first coiled segment 820 by the application of a mechanical stress in direction 824.

FIG. 8B depicts a perspective view of clip 800 in the stressed state after segment 822 has been passed within segment 820. Similar to the embodiments described above, the deformation of clip 800 places body 806 in torsion about longitudinal axis 810. This torsional force biases clip 800 towards the relaxed state, and allows clip 800 to at least partially close and preferably seal the PFO. Although not depicted here, the surface of clip 800 can have a roughened or raised texture to more adequately engage the tissue flaps. Clip 800 can be used to close a PFO in a method similar to that depicted with regard to clip 104 in FIGS. 5A-E. FIG. 8C depicts a perspective view of clip 800 deployed over two tissue flaps of a PFO.

The layout and shape of clip 800 provides certain advantages over the use of clip 104. For instance, coiled clip 800 can potentially close a PFO more easily than a clip 104 formed from the same material, due to the increased size and corresponding increased closing strength. This can be advantageous if the tissue flaps are relatively large and/or spaced farther apart. Because coiled segments 820 and 822 contact a greater surface area on the tissue flaps, the closing force is distributed over a wider area of tissue than with a similarly sized embodiment of clip 104. This can reduce the mechanical pressure placed on the tissue flaps per unit of surface area, allowing blood to be more easily circulated within the tissue flaps. However, the coiled configuration also exposes more surface area of clip 800 to the body, increasing the risk of bleeding. In environments where bleeding is a significant concern, the use of clip 104 can then be preferred.

FIG. 9 depicts a perspective view of another exemplary embodiment where clip 800 lies substantially within the X-Y plane while in the relaxed state. First coiled segment 820 and second coiled segments 822 are both concentrically curved about central axis 811. To deform clip 800 to the stressed state, a mechanical stress is applied such that each segment moves laterally away from the other placing body 806 in torsion about longitudinal axis 810.

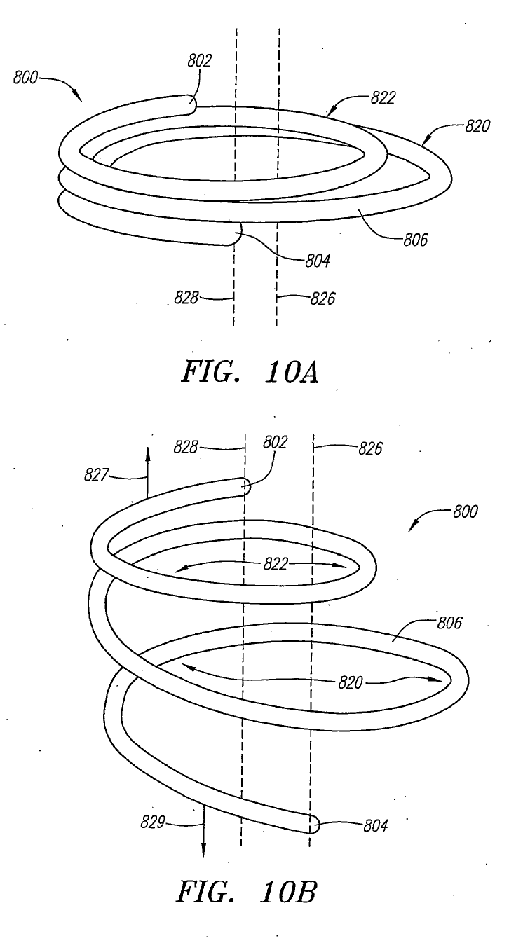

FIG. 10A depicts a perspective view of yet another exemplary embodiment of clip 800, while in the relaxed state. As opposed to the embodiments depicted above, here, coiled segments 820 and 822 are eccentric, i.e., each segment 820 and 822 is curved about a different axis. In this embodiment, first coiled segment 820 is coiled around central axis 826, while second coiled segment 822 is coiled around central axis 828. FIG. 10B depicts a perspective view of clip 800 in a partially stressed state after a mechanical stress is applied to deform ends 802 and 804 in directions 827 and 829, respectively.

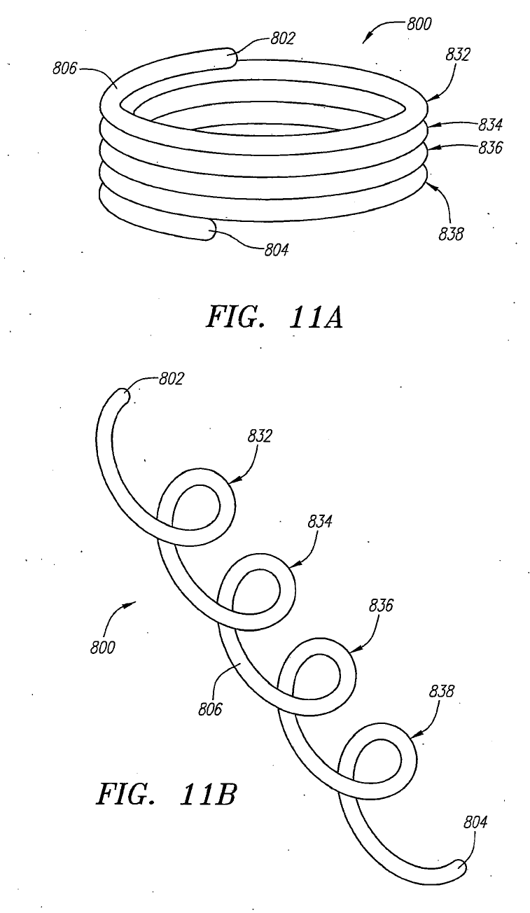

It should be noted that each coiled segment can loop less than or greater than 360 degrees about one or more axes, the actual length of each coiled segment being chosen based on the needs of the application. In addition, clip 800 can include numerous coiled segments. FIG. 11A depicts another exemplary embodiment of a concentrically shaped clip 800 having four coiled segments 832, 834, 836 and 838 of equal size. Here, clip 800 is shown in the relaxed state. FIG. 11B depicts clip 800 straightened from the relaxed state to the stressed state. During deployment, any number of coiled segments 832-838 can be placed on each side of the PFO. Preferably, the same number of coiled segments 832-838 are placed on each side; however, this can vary according to the elastic strength of each segment as well as the needs of the application and the manner of delivery.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the spirit of the disclosure. Furthermore, it should also be understood that the features or characteristics of any embodiment described or depicted herein can be combined, mixed or exchanged with any other embodiment.

What is claimed is:

1. A method of closing a patent foramen ovale having a septum primum and a septum secundum, comprising: delivering an elongate body having a proximal end and a distal end to the patent foramen ovale, the elongate body having a tissue piercing structure at its distal end and a coil releasably engaged with the elongate body; advancing the tissue piercing structure and the coil through the septa of the patent foramen ovale; and releasing the coil from the elongate body and withdrawing the tissue piercing structure from the septa of the patent foramen ovale, wherein the coil when released contracts to pinch the septum primum and the septum secundum together.

2. The method of claim 1, wherein the elongate body includes an opening near its distal end.

3. The method of claim 2, wherein the coil has a distal end that releasably engages the opening in the elongate body near its distal end.

4. The method of claim 3, wherein a loading portion releasably engages a proximal end of the coil, the coil being advanced through the patent foramen ovale while the coil is engaged with both the loading portion and the opening near the distal end of the elongate body to axially elongate and radially reduce the coil.

5. The method of claim 1, further comprising delivering a loading collar with the elongate body to the patent foramen ovale, the loading collar releasably engaging a proximal end of the coil.

6. The method of claim 5, wherein the elongate body is rotatable relative to the loading collar.

7. The method of claim 5, wherein the elongate body is axially slideable relative to the loading collar.

8. The method of claim 5, wherein the elongate body is advanced relative to the loading collar prior to advancing the coil to axially elongate the coil.

9. The method of claim 1, wherein the elongate body is delivered through an outer catheter.

10. The method of claim 1, wherein the tissue piercing structure and the coil are delivered first through the septum secundum and then through the septum primum.

11. The method of claim 1, wherein the coil is a first coil, and further comprising, after releasing the first coil from the elongate body and withdrawing the tissue piercing structure from the septa of the patent foramen ovale: advancing the tissue piercing structure and a second coil releasably engaged with the elongate body through the septa of the patent foramen ovale at a location adjacent to the first coil; and releasing the second coil from the elongate body and withdrawing the tissue piercing structure from the septa of the patent foramen ovale, wherein the second coil when released contracts to pinch the septum primum and the septum secundum together.

12. A method of closing a patent foramen ovale having a septum primum and septum secundum, comprising advancing a plurality of coils at least partially through the septa of the patent foramen ovale to secure the septum primum and septum secundum together.

13. The method of claim 12, wherein the plurality of coils are advanced sequentially through a single catheter.

14. The method of claim 12, wherein the plurality of coils are each advanced first through the septum secundum and then through the septum primum.

15. The method of claim 12, wherein the plurality of coils are each advanced first through the septum primum and then through the septum secundum.

16. The method of claim 12, wherein each of the coils is provided over a single elongate body and is advanced through the patent using a tissue piercing structure on the distal end of the elongate body.

17. The method of claim 12, wherein each of the coils after being advanced through the septa of the patent foramen ovale has a first end in the septum primum and a second end in the septum secundum.

18. The method of claim 12, wherein each of the coils after being advanced through the septa of the patent foramen ovale has a first end in the left atrium and a second end in the right atrium.

19. The method of claim 12, comprising advancing at least three coils through the septa of the patent foramen ovale.

20. An assembly for delivering a coil through tissue in a patient, comprising: a loading portion adapted to releasably engage a proximal end of the coil; and a tissue piercing structure adapted to releasably engage a distal end of the coil, wherein the loading portion holds the coil relative to the tissue piercing structure to axially elongate and radially reduce the coil.

21. The assembly of claim 20, wherein the loading portion is integral with the tissue piecing structure.

22. The assembly of claim 21, wherein the loading portion comprises a slot adapted to receive the proximal end of the coil.

23. The assembly of claim 21, wherein the tissue piercing structure includes an opening adapted to releasably engage the distal end of the coil.

24. The assembly of claim 20, wherein the loading portion comprises a loading collar, and the tissue piecing structure is moveable relative to the loading collar to axially advance and rotate the distal end of the coil relative to the proximal end of the coil to axially elongate the coil.

25. The assembly of claim 24, wherein the tissue piercing structure is provided on an elongate body having a proximal end and a distal end, the elongate body extending through the loading collar.

26. The assembly of claim 20, further comprising a coil having a proximal end releasably engaging the loading portion and a distal end releasably engaging the tissue piercing structure.

27. The assembly of claim 26, wherein the proximal end of the coil comprises a tang that extends into a diameter defined by the coil.

28. The assembly of claim 26, wherein the distal end of the coil comprises a tang that extends into a diameter defined by the coil.

29. The assembly of claim 20, wherein the coil is sized to extend through a septum primum and a septum secundum of a patent foramen ovale.

30. The assembly of claim 20, wherein the loading portion is adapted to releasably engage a plurality of coils.

31. A method of closing a patent foramen ovale having a septum primum and a septum secundum and a tunnel extending therebetween, comprising: positioning a countertraction element on one side of the patent foramen ovale; and delivering a closure device from the other side of the patent foramen ovale, the closure device adapted to hold the septum primum and septum secundum together, the closure device being advanced into position while the countertraction element holds the position of at least one of the septa.

32. The method of claim 31, wherein positioning a countertraction element on one side of the patent foramen ovale comprises positioning the countertraction element in a left atrium of a patient.

33. The method of claim 31, wherein positioning a countertraction element on one side of the patent foramen ovale comprises delivering a balloon to said one side of the patent foramen ovale.

34. The method of claim 33, wherein the balloon is delivered on a distal end of a catheter.

35. The method of claim 34, wherein the balloon is delivered through the tunnel of the patent foramen ovale.

36. The method of claim 34, wherein the balloon is delivered by penetrating through the septa of the patent foramen ovale.

37. The method of claim 31, wherein positioning a countertraction element on one side of the patent foramen ovale comprises expanding a wire on one side of the patent foramen ovale.

38. The method of claim 31, wherein positioning a countertraction element on one side of the patent foramen ovale comprises delivering a guide wire through said tunnel to said one side of the patent foramen ovale.

39. The method of claim 38, wherein the guide wire has a generally S-shaped distal end.

40. The method of claim 38, comprising delivering at least one closure device through the septa of the patent foramen ovale adjacent the guide wire.

41. The method of claim 31, wherein the countertraction element is first delivered to said other side of the patent foramen ovale, and is then advanced to said one side of the patent foramen ovale.

42. The method of claim 31, wherein the countertraction element is delivered directly to said one side of the patent foramen ovale.

43. The method of claim 31, wherein the closure device penetrates at least partially through the septa of the patent foramen ovale.

44. The method of claim 31, wherein the closure device extends at least partially through the tunnel of the patent foramen ovale.

45. The method of claim 31, further comprising securing the countertraction element at the patent foramen ovale with the closure device.

46. The method of claim 31, wherein the countertraction element includes a cover, and the closure device is delivered through the cover.

47. A method of closing an opening in a patient, comprising: positioning a countertraction element relative to said opening to hold said opening in place; and delivering a closure device to the opening while said countertraction element holds said opening in place.

48. The method of claim 47, wherein the opening is a patent foramen ovale.

49. The method of claim 47, wherein positioning a countertraction element relative to said opening comprises positioning a removable implant within the opening.

50. The method of claim 47, wherein the closure device further secures the countertraction element relative the opening.

51. The method of claim 47, further comprising removing the countertraction element after delivering the closure device.
